(19) United States
(12) Patent Application Publication
(10) Pub. No.: US 2026/0018247 A1
(43) Pub. Date: Jan. 15, 2026

(54) SYSTEMS AND METHODS OF DETERMINING A NUCLEIC ACID SEQUENCE BASED ON MUTATED SEQUENCE READS

(71) Applicant: Illumina, Inc., San Diego, CA (US)

(72) Inventors: Aaron Earl Darling, Hornsby (AU); Aaron L. Halpern, San Carlos, CA (US)

(21) Appl. No.: 18/868,645

(22) PCT Filed: Mar. 5, 2024
(86) PCT No.: PCT/US2024/018539

§ 371 Date: Nov. 22, 2024

Related U.S. Application Data
(60) Provisional application No. 63/489,550, filed on Mar. 10, 2023.

(51) Int. Cl. G16B 30/10; G16B 30/20

(52) U.S. Cl. CPC G16B 30/10; G16B 30/20

(57) ABSTRACT

Described herein are methods and systems for determining a sequence of a nucleic acid template by removing mutations found in a mutated sequence read. In some embodiments, methods and systems include steps of aligning unmutated sequence reads of a nucleic acid template to mutated sequence reads, and determining a most probable sequence of the nucleic acid template and a per-base accuracy score correlated with the probability that the base matches with the nucleic acid template, thereby determining the sequence of the nucleic acid template.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims priority to U.S. Provisional Patent Application No. 63/489,550, filed Mar. 10, 2023, which is hereby incorporated by reference in its entirety.

### BACKGROUND

### Field

The present disclosure relates generally to the field of sequencing nucleic acids. More specifically, the present disclosure relates to the field of determining the sequence of a nucleic acid template by removing mutations found in a mutated sequence read.

### Description

Conventional nucleotide sequence analysis techniques known as “error correction” or “polishing” are used for correcting errors that were unintentionally introduced into the sequencing process by noisy or error-prone measurement of the primary sequence signal. However, such techniques have been criticized as not being effective, particularly through repeat regions. In some instances, employment of such methods produces an “average sequence” of copies of repeat regions, rather than an accurate representation of any specific copy of a nucleic acid template containing the repeat region.

### SUMMARY

The methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the systems and methods disclosed herein provide advantages over other known systems and methods.

Described herein are computer-implemented methods of determining a sequence of a nucleic acid template by removing mutations found in a mutated sequence read. In some embodiments, the method includes aligning unmutated sequence reads of a nucleic acid template to mutated sequence reads, wherein the mutated sequence reads comprise one or more mutations, and determining a most probable sequence of the nucleic acid template and for each base of the most probable sequence, a per-base accuracy score correlated with the probability that the base matches with the nucleic acid template; thereby determining the sequence of the nucleic acid template.

In some embodiments, the mutated sequence reads are of a mutated nucleic acid template. In some embodiments, the one or more mutations comprise mutations introduced during sample preparation. In some embodiments, the one or more mutations comprise mutations which were randomly introduced during sample preparation by mutagenesis. In some embodiments, the one or more mutations comprise mutations which were unintentionally introduced. In some embodiments, the nucleic acid template is longer than the mutated sequence reads and the unmutated sequence reads.

In some embodiments, determining a most probable sequence of the nucleic acid template includes determining a most probable base for each site associated with the alignment of the unmutated sequence reads to the mutated sequence reads. In some embodiments, determining a most probable sequence of the nucleic acid template includes determining a most probable sequence of a subset of sites associated with the alignment of the unmutated sequence reads to the mutated sequence reads.

In some embodiments, determining a most probable sequence of the nucleic acid template and for each base of the most probable sequence, a per-base accuracy score includes, on a site-by-site basis, summarizing the probability of each base at each site associated with the unmutated sequence reads.

In some embodiments, determining a most probable sequence of the nucleic acid template and for each base of the most probable sequence, a per-base accuracy score includes incorporating information related to the probability of a mutation at each site of a mutated sequence read.

In some embodiments, determining a most probable sequence of the nucleic acid template and for each base of the most probable sequence, a per-base accuracy score includes incorporating information that describes a probability of an error associated with a sample preparation or sequencing process.

In some embodiments, determining a most probable sequence of the nucleic acid template and for each base of the most probable sequence, a per-base accuracy score includes determining a most probable base for a site of a mutated sequence read that is not aligned to an unmutated sequence read, based on a statistically inferred probability of a mutation at the site.

In some embodiments, determining a most probable sequence of the nucleic acid template and for each base of the most probable sequence, a per-base accuracy score includes incorporating information that describes variation in mutation rate between mutated sequence reads or unequal mutation rates of adenine, cytosine, guanosine, and thymine.

In some embodiments, the nucleic acid template is derived from a nucleic acid sample comprising two or more copies of a repeat region of the nucleic acid template.

In some embodiments, determining a most probable sequence of the nucleic acid template and for each base of the most probable sequence, a per-base accuracy score includes estimating a probability that an unmutated sequence read was derived from a same copy of a repeat region as a mutated sequence read to which the unmutated sequence read is aligned.

In some embodiments, determining a most probable sequence of the nucleic acid template and for each base of the most probable sequence, a per-base accuracy score includes applying an optimization process, wherein the optimization process iteratively updates an estimate of the probability that an unmutated sequence read was derived from a same copy as the mutated sequence read to which the unmutated sequence read is aligned, while updating an inference of a most probable base. In some embodiments, the optimization process comprises applying an Expectation Maximation method. In some embodiments, the optimization process incorporates information that describes variations in read length.

In some embodiments, a contribution of each unmutated sequence read to a probability calculation of a most probable base is weighted by a weighting scheme. In some embodiments, the weighting scheme is related to sequence identity of the unmutated sequence read and a current iterative estimate of the sequence of the nucleic acid template.

In some embodiments, determining a most probable sequence of the nucleic acid template and for each base of the most probable sequence, a per-base accuracy score includes selecting a subset of unmutated sequence reads among unmutated sequence reads derived from different copies of the repeat region. In some embodiments, selecting a subset of unmutated sequence reads comprises scoring alignments of unmutated sequence reads to the mutated sequence reads and selecting top-scoring alignments.

In some embodiments, the method includes determining a sequence of a copy of a repeat region of the nucleic acid template. In some embodiments, the two or more copies of a repeat region include two or more haplotypes, and the method includes determining a sequence of a haplotype of the nucleic acid sample.

In some embodiments, the per-base accuracy score includes a per-base Quality Value (QV). In some embodiments, the method further includes determining an overall accuracy score related to the most probable sequence.

In some embodiments, the method further includes, prior to aligning unmutated sequence reads of a nucleic acid template to mutated sequence reads, assembling the unmutated sequence reads. In some embodiments, aligning unmutated sequence reads of a nucleic acid template to mutated sequence reads includes aligning the mutated sequence reads to the assembly of unmutated sequence reads.

In some embodiments, the method further includes, prior to aligning unmutated sequence reads of a nucleic acid template to mutated sequence reads, assembling the mutated sequence reads. In some embodiments, aligning unmutated sequence reads of a nucleic acid template to mutated sequence reads includes aligning the unmutated sequence reads to the assembly of mutated sequence reads.

In some embodiments, the method further includes, prior to aligning unmutated sequence reads of a nucleic acid template to mutated sequence reads, aligning the unmutated sequence reads or the mutated sequence reads to a reference sequence.

In some embodiments, the method further includes, prior to aligning unmutated sequence reads of a nucleic acid template to mutated sequence reads, assembling the mutated sequence reads, generating a consensus of the assembled mutated sequence reads, and aligning the unmutated sequence reads to the consensus. In some embodiments, aligning unmutated sequence reads of a nucleic acid template to mutated sequence reads includes replacing the consensus with the mutated sequence reads in said alignment of the unmutated sequence reads to the consensus. In some embodiments, assembling the mutated sequence reads includes aligning the mutated sequence reads to a reference sequence.

In some embodiments, the method further includes sequencing unmutated nucleic acid template molecules and mutated nucleic acid template molecules to obtain unmutated sequence reads and mutated sequence reads.

In some embodiments, the method includes aligning the unmutated sequence reads and the mutated sequence reads to a reference sequence; determining an initial rendered sequence by determining a most probable base for each site associated with the alignment of the unmutated sequence reads to the mutated sequence reads, incorporating information that describes variation in mutation rate between mutated sequence reads or unequal mutation rates of adenine, cytosine, guanosine, and thymine; and determining a further rendered sequence by comparing k-mers from the initial rendered sequence to k-mers from unmutated sequence reads.

In some embodiments, the method includes aligning the unmutated sequence reads and the mutated sequence reads to a reference sequence; realigning unmutated sequence reads that have a mapping location that overlaps a mutated sequence read directly to the mutated sequence read; scoring alignments of unmutated sequence reads to the mutated sequence reads and selecting top-scoring alignments; and determining a rendered sequence by determining a most probable base for each site associated with the alignment of the unmutated sequence reads to the mutated sequence reads.

In some embodiments, the method includes determining a rendered sequence by: determining a most probable base for each site associated with the alignment of the unmutated sequence reads to the mutated sequence reads, and applying an Expectation Maximation (EM) optimization process, wherein the EM optimization process iteratively updates an estimate of the probability that an unmutated sequence read was derived from a same copy as the mutated sequence read to which the unmutated sequence read is aligned, while updating an inference of the most probable base.

In some embodiments, the method includes generating a consensus of the assembled mutated sequence reads; aligning the unmutated sequence reads to the consensus; replacing the consensus with the mutated sequence reads in said alignment of the unmutated sequence reads to the consensus; and determining a rendered sequence by: determining a most probable base for each site associated with the alignment of the unmutated sequence reads to the mutated sequence reads, and applying an Expectation Maximation (EM) optimization process, wherein the EM optimization process iteratively updates an estimate of the probability that an unmutated sequence read was derived from a same copy as the mutated sequence read to which the unmutated sequence read is aligned, while updating an inference of the most probable base.

In another aspect, disclosed herein are systems comprising a processor configured to perform a method. In some embodiments, the method includes aligning unmutated sequence reads of a nucleic acid template to mutated sequence reads of a mutated nucleic acid template, wherein the mutated sequence reads comprise one or more mutations, and determining a most probable sequence of the nucleic acid template and for each base of the most probable sequence, a per-base accuracy score correlated with the probability that the base matches with the nucleic acid template; thereby determining the sequence of the nucleic acid template.

In a further aspect, disclosed herein are computer readable mediums. In some embodiments, the computer readable medium comprises instructions that when executed by a processor perform a method comprising: aligning unmutated sequence reads of a nucleic acid template to mutated sequence reads of a mutated nucleic acid template, wherein the mutated sequence reads comprise one or more mutations, and determining: a most probable sequence of the nucleic acid template, and for each base of the most probable sequence, a per-base accuracy score correlated with the probability that the base matches with the nucleic acid template; thereby determining the sequence of the nucleic acid template.

### BRIEF DESCRIPTION OF THE DRAWINGS

FIG. 1 depicts an embodiment of a method for determining a sequence of a nucleic acid template by removing mutations found in a mutated sequence read.

FIG. 2 depicts an embodiment of a system including a processor configured to perform a method for determining a sequence of a nucleic acid template by removing mutations found in a mutated sequence read.

FIG. 3 depicts an example of determining the sequence of a nucleic acid template using mutated sequence reads and unmutated sequence reads.

FIG. 4 depicts an example of determining the sequence at a site that contains a heterozygous transition SNP.

FIG. 5 depicts an example of determining the sequence of a real human genome dataset.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

Some embodiments relate to systems and methods for determining the nucleotide sequence of a nucleic acid template. In some cases, sequencing the nucleic acid template may involve introducing random mutations into the template so that it can be fragmented into smaller sequences which are more easily run through a next generation sequencing process. Incorporation of the random mutations can help to reconstruct the sequence of a mutated nucleic acid template by alignment of the mutated fragments with each other. However, in some cases, the sequence of the original, unmutated, template molecule is desired. Embodiments relate to methods of determining the unmutated sequence of the original nucleic acid template.

One embodiment of determining the unmutated sequence of the original nucleic acid template can involve fragmenting the unmutated nucleic acid template and determining the sequences of the unmutated fragments. The system and method may then align the unmutated sequence reads from the nucleic acid template to mutated sequence reads of the nucleic acid template to determine a most probable sequence of the unmutated nucleic acid template. This process can include, for each base of the most probable unmutated nucleic acid template, determining a per-base accuracy score correlated with the probability that the base matches with the nucleic acid template. By using this per-base accuracy score, the most likely sequence of the unmutated nucleic acid template may be determined. This is described in more detail in the following sections.

### Definitions

As used herein, “align,” “alignments,” or “aligning” refers to arranging two or more nucleic acid sequences to identify regions of similarity.

“Nucleic acid template” can be any nucleic acid molecule that the user would like to sequence. A “nucleic acid template” can be single-stranded or can be part of a double-stranded complex. If the nucleic acid template is made up of deoxyribonucleotides, it may form part of a double-stranded DNA complex. In this case, one strand (for example the coding strand) may be considered to be the nucleic acid template, and the other strand is a nucleic acid molecule that is complementary to the nucleic acid template. The nucleic acid template may be a DNA molecule corresponding to a gene, may comprise introns, may be an intergenic region, may be an intragenic region, may be a genomic region spanning multiple genes, or may, indeed, be an entire genome of an organism. A nucleic acid template may include a repetitive sequence, for example, a variable number tandem repeat (VNTR) or short tandem repeat (STR). A nucleic acid template may be derived from a nucleic acid sample.

A “nucleic acid sample” includes any sample of nucleic acids. The nucleic acid sample may be a sample of nucleic acids derived from a human, for example a sample extracted from a skin swab of a human patient. The nucleic acid sample may be a sample of nucleic acids derived from a nonhuman. A nucleic acid sample may be derived from sources such as a sample from a water supply. Such a sample could contain hundreds, thousands, millions or even billions of nucleic acid template molecules. A nucleic acid sample may include a single genome, or a nucleic acid sample may include multiple non-identical genomes (such as from different organisms or different species or strains). A nucleic acid sample may contain multiple haplotypes from a single organism.

As used herein, “repeat region” refers to a region within a nucleic acid template, wherein the nucleotide sequence of that region is copied and present in at least one other instance in a nucleic acid sample. In other words, a nucleic acid sample may have two or more copies of a repeat region of a nucleic acid template. In some embodiments, two copies of a repeat region may be non-identical, for example containing single base differences. In some embodiments, a repeat region contains a repetitive sequence (for example, a VNTR or STR) such as wherein the repeats are adjacent or tandem with one another. For example, a nucleic acid sample may include a nucleic acid template having a repeat region that includes a repetitive sequence, and the nucleic acid sample may include another copy of the repeat region with repetitive sequences elsewhere (for example, on another chromosome, or a different location on the same chromosome). As another example, in some embodiments, a repeat region may include the full length of a chromosome. In such an embodiment, a “copy” of the repeat region would be another copy of the chromosome found within the nucleic acid sample, such as a second haplotype.

“Mutated nucleic acid template” refers to a nucleic acid template that includes one or more mutations, such as mutated bases, as compared to the original sequence of the nucleic acid template. In other words, a mutated nucleic acid template refers to a mutated copy of the nucleic acid template.

As used herein, “mutation” refers to a change in a nucleic acid sequence. For example, a mutation includes a substitution of one nucleotide base for another. A mutation may be a transition mutation (from A to G, G to A, C to T, or T to C). In another embodiment, the mutation is a transversion mutation. In some embodiments, mutations are intentionally introduced into a nucleic acid via a sample processing step which randomly introduces mutations into the nucleic acid molecule. In some embodiments, the mutations are unintentionally introduced, such as by error or unintended consequence during sample preparation or library preparation. In some embodiments, mutations are caused by unintentional errors during sequencing of the nucleic acid template, such as base calling errors.

As used herein, a “mutated sequence read,” also referred to herein as “dream,” or “dream sequence,” refers to a sequence read that contains mutations as compared to the nucleic acid template. In some embodiments, a mutated sequence read is a sequence read of a mutated nucleic acid template. In some embodiments, a mutated sequence read is obtained by sequencing a region of a mutated nucleic acid template. In some embodiments, a mutated sequence read is obtained by sequencing a region of a nucleic acid template and contains mutations due to sequencing error. A mutated sequence read may have a length that is less than the length of a nucleic acid template. In some embodiments, a mutated sequence read has a length of about 100 to 600 nt, for example about 150 nt or about 300 nt. In some embodiments, mutated sequence reads may be assembled by aligning mutated sequence reads. Any method known in the art may be used for such an alignment, for example, methods as described in US20210174905A1.

As used herein, “unmutated sequence read” refers to a sequence read of the nucleic acid template. In some embodiments, an unmutated sequence read is obtained by sequencing a region of a nucleic acid template. An unmutated sequence read may have a length that is less than the length of a nucleic acid template, for example, a length of about 100 to 600 nt, for example about 150 nt or about 300 nt.

“Render” or “rendering” refers to a process of determining a most probable sequence of a nucleic acid template using alignments of unmutated sequence reads and mutated sequence reads. In some embodiments, rendering includes determining a most probable sequence of a nucleic acid template by replacing a mutation in a mutated sequence read with a most probable base.

“Most probable base” refers to the nucleic acid base which is calculated to have the highest probability of matching the nucleic acid template (in some cases, the specific copy of a nucleic acid template) from which a mutated sequence read derives. For example, the most probable base may be a maximum a posteriori base as described herein.

“Most probable sequence” refers to a nucleic acid sequence that is calculated to have the highest probability of matching with a nucleic acid template (in some cases, the specific copy of a nucleic acid template) from which a mutated sequence read ultimately derives (such as the unmutated nucleic acid template as existed before mutations were introduced). Thus, in some embodiments, a most probable sequence does not include (is statistically predicted to not include) mutations. Thus, in some embodiments, a most probable sequence may be produced by statistically removing mutations from a mutated sequence read to estimate the sequence of the (unmutated) nucleic acid template.

### Methods of Determining a Sequence of a Nucleic Acid Template Using Mutated Sequence Reads and Unmutated Sequence Reads

Embodiments relate to methods for determining a sequence of a nucleic acid template molecule using a sample processing step that intentionally introduces random mutations into a copy of the sample nucleic acid. For example, methods for introducing random mutations into a target nucleic molecule are described in U.S. Patent Application Publication No. US20210010008A1. In some embodiments, mutations are introduced by the Illumina® Complete Long Read sequencing sample prep chemistry. The introduction of such random mutations may be useful in sequence assembly, for example, as described in U.S. Patent Application Publication No. US20210174905A1. For example, the creation of a mutated copy of the nucleic acid template may be particularly useful for performing nucleic acid sequencing when a nucleic acid template includes repeat regions, for example when a nucleic acid sample includes two or more non-identical copies (for example, haplotypes) of a repeat region.

Accordingly, some embodiments of systems and methods disclosed herein include steps of obtaining one or more nucleic acid samples (including obtaining one nucleic acid sample that is split into two nucleic acid samples and including two nucleic acid samples from the same subject). Some embodiments include steps of randomly introducing mutations into nucleic acid molecules in one or more nucleic acid samples, producing a mutated nucleic acid sample. Some embodiments include keeping a nucleic acid sample unmutated by not randomly introducing mutations into nucleic acid molecules in the nucleic acid sample. Some embodiments herein also include steps of sequencing the nucleic acid molecules (such as a nucleic acid template) of the (unmutated) nucleic acid sample, producing unmutated sequence reads. Some embodiments disclosed herein also include sequencing the nucleic acid molecules (such as a mutated nucleic acid template) of the mutated nucleic acid sample, producing mutated sequence reads. Sequencing of mutated and unmutated nucleic acid sample may be performed simultaneously or sequentially. Sequencing may be accomplished by any method known to those of skill in the art, including next-generation sequencing (NGS).

While mutated sequence reads are useful in determining the sequence of a nucleic acid template, it may also be desirable to determine the sequence of the original nucleic acid template molecule as it existed prior to the introduction of random mutations. To accomplish this, mutated sequence reads may be aligned to unmutated sequence reads to determine the sequence of the nucleic acid template in a process denoted herein as “rendering.”

However, determining the alignments of the mutated sequence reads with the unmutated sequence reads may be ambiguous in some situations. For example, when the nucleic acid template has multiple copies of repeat regions, such as repeat regions containing repetitive sequences, and/or when a nucleic acid sample contains two or more haplotypes of a nucleic acid template, alignments of unmutated sequence reads to mutated sequence reads may be ambiguous because the mutated sequence reads may align at many different positions on the nucleic acid template.

The present disclosure relates to systems and methods that, given one or more mutated sequence reads and one or more unmutated sequence reads from the same nucleic acid sample, can accurately predict the sequence of a nucleic acid template. In some embodiments, the methods involve aligning the mutated sequence reads and the unmutated sequence reads and statistically determining a most probable sequence of the nucleic acid template and a per-base accuracy score corresponding to each base of the most probable sequence.

Described herein are various computer-implemented methods of determining the correct sequence of a nucleic acid template by removing mutations found in a mutated sequence read. In some embodiments, the mutated sequence reads include one or more nucleic acid mutations. In some embodiments, the mutated sequence reads are of a mutated nucleic acid template. In some embodiments, the one or more mutations comprise mutations introduced during sample preparation. In some embodiments, the one or more mutations were randomly introduced during sample preparation by mutagenesis. In some embodiments, the mutations include intentionally introduced nucleic acid mutations. The method of introducing such mutations can be any method known in the art. In some embodiments, the mutations include substitution mutations. In some embodiments, the mutations include transition mutations.

In some embodiments, the nucleic acid template is longer than the mutated sequence reads and/or the unmutated sequence reads. For example, in some embodiments, the unmutated sequence reads and mutated sequence reads are greater than 10 bp, greater than 100 bp, less than 200 bp, less than 300 bp, less than 400 bp, less than 500 bp, less than 600 bp, less than 700 bp, less than 1,000 bp, less than 3,000 bp, or within a range constructed from any of the aforementioned values. In some embodiments, the unmutated sequence reads and mutated sequence reads are approximately 300 bp in length. In some embodiments, the unmutated sequence reads and mutated sequence reads are approximately 150 bp in length.

In some embodiments, the methods described herein are used to remove mutations that have been unintentionally introduced into the sequencing process, such as by noisy or error-prone measurement of the primary sequence signal. In such an embodiment, the methods described herein may be considered improved methods of error correction or polishing.

Alignment to a Reference Sequence and/or Consensus Sequence

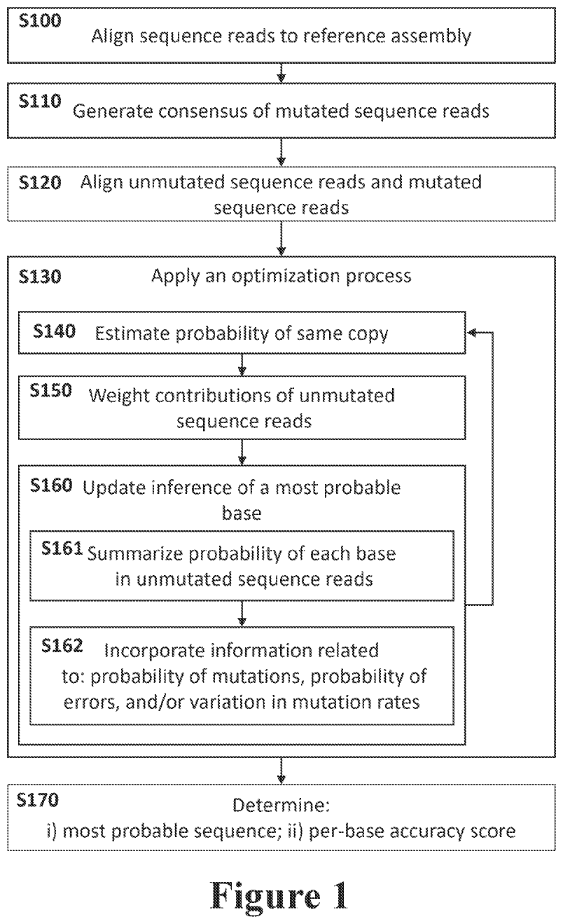

Some embodiments include systems or methods of determining a sequence of a nucleic acid template by removing mutations found in a mutated sequence read by the method shown in FIG. 1. As illustrated, the method begins at a step S100 wherein sequence reads, such as unmutated sequence reads and/or mutated sequence reads, are aligned to a reference sequence.

Alignment may be performed by methods known to those of skill in the art. The reference sequence may be derived from a different nucleic acid sample than the nucleic acid template or may be from the same nucleic sample. In some embodiments, the reference sequence is from the same species of an organism as the sample nucleic acid template. In some embodiments, the reference sequence is a publicly available reference sequence.

In some embodiments, the systems and methods further comprise a step S110 of assembling the mutated sequence reads, generating a consensus of the assembled mutated sequence reads, and aligning the unmutated sequence reads to the consensus.

Previous work on Sequence Analysis by Mutagenesis developed techniques to accurately call consensus sequences from a set of mutated sequences, for example those disclosed in Keith J M, Adams P, Bryant D, Cochran D A E, Lala G H, Mitchelson K R. Algorithms for sequence analysis via mutagenesis. Bioinformatics. 2004 05; 20 (15): 2401-2410. doi: 10.1093/bioinformatics/bth258. To determine a most probable sequence of a nucleic acid template, previous approaches would construct a multiple alignment of the mutated sequence reads, either via mapping to a reference sequence or via a de novo multiple alignment method, call the consensus, and copy the consensus sequence into each of the corresponding sites of aligned mutated sequence reads, thereby determining a most probable sequence of the nucleic acid template. When a reference sequence is used, it can either be independently constructed, or constructed from unmutated sequence reads, or constructed from mutated sequence reads.

In some embodiments, instead of using the consensus directly to determine a most probable base or a most probable sequence, the consensus of mutated sequence reads is instead used to improve the accuracy of alignment of unmutated sequence reads to mutated sequence reads. Briefly, in step S110, the consensus of the mutated sequence reads is called, for example, using a method disclosed in Keith et al. (2004), to produce a consensus of mutated sequence reads. Further in step S110, the consensus is then used as a mapping target for the unmutated sequence reads. Further in step S110, the alignments onto the consensus are then translated back onto the original (non-consensus) mutated sequence reads, and the alignments of unmutated sequence reads to the original mutated sequence reads are used in the methods of determining a sequence described herein.

One advantage of this approach over performing an alignment of unmutated sequence reads directly to the original mutated sequence reads is that the consensus may have much higher sequence similarity to the unmutated sequence reads that are derived from the same haplotype. As further described herein, a nucleic acid sample from which a nucleic acid template is derived may include multiple copies of a repeat region, such as multiple haplotypes, which may make alignments of unmutated sequence reads to mutated sequence reads ambiguous or incorrect. The higher sequence identity means that mapping and alignment is more accurate, faster to compute, and may contain fewer unmutated sequence reads which become aligned to mutated sequence reads from another copy of a repeat region (such as another haplotype). An advantage of this approach over using the consensus method for determining a most probable sequence is that use of the unmutated sequence reads can provide much higher confidence and accuracy in the resulting basecalls.

### Alignment of Mutated Sequence Reads and Unmutated Sequence Reads

In some embodiments, the methods and systems described herein further include a step S120 of aligning unmutated sequence reads of a nucleic acid template to mutated sequence reads. The mechanism to produce such alignments can be any method known to one of skill in the art.

In some embodiments, after the step S110 of generating a consensus sequence, the step S120 alignment of the unmutated sequence reads to the mutated sequence reads proceeds by replacing the consensus with the mutated sequence reads in the alignment of the unmutated sequence reads to the consensus. In some embodiments, a consensus sequence is not generated, and unmutated sequence reads and mutated sequence reads are aligned by mapping to each other by methods known to those of skill in the art.

In some embodiments, the alignment of the mutated sequence reads to the unmutated sequence reads may include about a 30-fold coverage of the unmutated sequence reads. In some embodiments, the alignment includes about 10-fold coverage of unmutated sequence reads, about 15-fold coverage, about 20-fold coverage, about 30-fold coverage, about 40-fold coverage, about 50-fold coverage, about 60-fold coverage, or greater, or a range constructed from any of the aforementioned values. In some embodiments, the alignment includes coverage as described above, for example, 15-fold coverage, of an individual haplotype of a nucleic acid template sample, through the unmutated sequence reads.

In some embodiments, the method further includes, prior to the step S120 alignment, assembling the unmutated sequence reads to generate an assembly of unmutated sequence reads. In some embodiments, unmutated sequence reads are assembled with use of a reference sequence. In some embodiments, the step S120 of alignment of the unmutated sequence reads to the mutated sequence reads may include aligning the mutated sequence reads to the assembly of unmutated sequence reads.

In some embodiments, prior to the step S120 alignment of the unmutated sequence reads to the mutated sequence reads, the method includes assembling the mutated sequence reads to generate an assembly of mutated sequence reads. In some embodiments, the mutated sequence reads are assembled through the use of a reference sequence. In some embodiments, the step S120 alignment of the unmutated sequence reads to the mutated sequence reads may include aligning the unmutated sequence reads to the assembly of mutated sequence reads.

### Approaches to Multiple Copies of a Repeat Region

In some embodiments, the nucleic acid template is derived from a nucleic acid sample comprising two or more copies of a repeat region of the nucleic acid template. For example, a nucleic acid sample may include two or more haplotypes. As another example, a nucleic acid sample may include a repeat region that includes a sequence (such as a repetitive sequence) that has a copy in another location on nucleic acid molecules in the nucleic acid sample (such as, on another chromosome or a different region of the same chromosome, in other words a different location in a genome found within the nucleic acid sample). In some embodiments, each copy of the repeat region has slight variations from each other, such that the nucleic sample includes two or more non-identical copies of the repeat region.

A challenge faced by methods for determining the sequence of a target nucleic acid using mutated sequence reads and unmutated sequence reads is the uncertainty of whether any given unmutated sequence read covering a repeat region has been derived from the “same copy” of the repeat region as a mutated sequence read covering a repeat region. A “same copy” can be conceptualized as referring to where an error-free unmutated sequence read would be 100% identical to the homologous portion of the (unknown) error-free, mutated read sequence (with mutations removed) to which it has aligned. This challenge can be particularly difficult when the sequence reads cover long repetitive sequence areas of a nucleic acid template.

Further, a problem that arises due to mutagenesis (and error-prone sequencing) is that a particular change in a repeat region can alter the sequence of one repeat region to be more similar to another copy of the repeat region that may be present elsewhere in the nucleic acid sample (such as, on another chromosome or a different region of the same chromosome, or another haplotype). This will also contribute to ambiguity in alignments of unmutated sequence reads to mutated sequence reads.

When the above-described methods are applied in their simplest form, unmutated sequence reads covering one copy of a repeat region may align to mutated sequence reads covering another copy of the repeat region, introducing error. Alignment of unmutated sequence reads to the incorrect copy of a repeat region in mutated sequence reads may prevent accurate identification of mutated sites within a mutated sequence read, and may lead to a large number of low confidence (low QV) base calls (for example, bases having a low per-base accuracy score) in the most probable sequence.

Various exemplary approaches for determining a most probable sequence of a nucleic acid template, when the nucleic acid sample from which the nucleic acid template is derived includes two or more copies of a repeat region, are described below in further detail. For example, approaches to this problem may include a step S130 of applying an optimization process as further described herein, or a step of selecting a subset of unmutated sequence reads as further described herein.

In some embodiments, the method includes determining the sequence of a copy of a repeat region of the nucleic acid template. By way of nonlimiting example, systems and methods described herein may be used to determine the sequence of a copy of a repeat region in a nucleic acid template, where the repeat region (such as a repeat region containing a repetitive sequence) has another non-identical copy in another location (such as elsewhere in a genome) in the nucleic acid sample. In some embodiments, the two or more copies of a repeat region include two or more haplotypes, and the method includes determining the sequence of a haplotype of the nucleic acid sample. By way of nonlimiting example, a nucleic acid sample from a human may include two haplotypes. Embodiments of the systems and methods disclosed herein may advantageously determine the sequence of one or more haplotypes.

### Application of an Optimization Process

After the process has aligned unmutated sequence reads and mutated sequence reads at the step S120, the systems and methods can move to a step S130 of applying an optimization process (that includes steps S140, S150 and S160, which may be iterated). In some embodiments, in step S130, the optimization process iteratively updates an estimate of the probability that an unmutated sequence read was derived from a same copy as the mutated sequence read to which the unmutated sequence read is aligned, while updating an inference of a most probable base.

As previously described herein, a challenge that faces rendering, as well as related polishing or error correction techniques, is handling of repeat regions, where similar but non-identical copies of repeat regions exist in different abundances in a nucleic acid sample. For this reason, it may be advantageous to determine (or to estimate a probability) that an unmutated sequence read has been derived from the same copy of a repeat region as the mutated sequence read to which it has aligned, and to incorporate this estimate information in the rendering process so that sequence reads which may have derived from another copy of a repeat region contribute less to the final result. Various exemplary approaches, such as the step S130 of applying an optimization model, are described below. In some embodiments, a combination of any of these methods is used to estimate whether an unmutated sequence read has been derived from the same copy of a repeat region as a mutated sequence read.

This may be described by reference to the following non-limiting illustrative examples. In one example, there are two similar but non-identical repeat regions, r1 and r2, with the r2 sequence occurring in 10 places in a genome in a nucleic acid sample, while the r1 sequence occurs in only a single place. Alternatively, in another example in a metagenomic setting, r2 occurs in a species or strain that is at 10-fold higher abundance than the species or strain that contains r1, in the nucleic acid sample. In both such examples, the alignment of unmutated sequence reads to mutated sequence reads will typically produce alignments of the unmutated sequence reads from both r1 and r2 against the mutated sequence reads from r1 and r2. A problem occurs when determining the sequence of the nucleic acid template containing r1, because 10-fold more unmutated sequence reads from r2 have aligned (incorrectly) to the mutated sequence reads of r1, than unmutated sequence reads from r1. In the absence of a technique to select the correct unmutated sequence reads, those which derive from the same copy of the repeat region r1, the unmutated sequence reads from r2 would dominate the signal when rendering r1 and cause the predicted most probable sequence of r1 to take on the r2 sequence as a byproduct of rendering. This phenomenon has been reported by the academic community to occur when using standard error correction or assembly polishing techniques, leading to poor accuracy in repeat regions. However, techniques that can weight the contribution of unmutated sequence reads to rendering, such as those described herein, have the ability to select the correct reads to use for rendering in these situations.

By way of nonlimiting example, the method may include the step S140 of estimating a probability for each unmutated sequence read aligned to one or more mutated sequence reads, that the unmutated sequence read was generated from the same copy of a repeat region (such as the same haplotype) as the one or mutated sequence reads to which the unmutated sequence read is aligned. In some embodiments, the step S140 includes estimating a probability that an unmutated sequence read, which is aligned to one or more mutated sequence reads at a given site, was generated by sequencing a nucleic acid molecule that contains the same copy of a repeat region as the one or more mutated sequence reads to which the unmutated sequence read is aligned. In some embodiments, the unmutated sequence read covers the repeat region. For example, the probability that an unmutated sequence read was derived from the same haplotype as the mutated sequence read to which the unmutated sequence read is aligned, may be estimated in the step S140. Various statistical models may be used in the step S140 to estimate the probability that the unmutated sequence read was derived from a same copy of a repeat region as a mutated sequence read to which the unmutated sequence read is aligned. Thus, the systems and methods described herein may include the step S140 of estimating a probability that an unmutated sequence read was derived from a same copy of a repeat region as a mutated sequence read to which the unmutated sequence read is aligned.

In some embodiments, the method further includes the step S150, whereby a contribution of each unmutated sequence read to a probability calculation of a most probable base is weighted by a weighting scheme. For example, in the step S150, unmutated sequence reads aligned to mutated sequence reads may be given unequal weights when statistically inferring a most probable base. For example, if for a particular site in an alignment of unmutated sequence reads to mutated sequence reads, some unmutated sequence reads have the base “G” and some have the base “A,” unmutated sequence reads which have the base “G” may be given more weight when statistically inferring a most probable base for the site.

In some embodiments, in the step S150, the weighting scheme is related to sequence identity of the unmutated sequence read and a current iterative estimate of the sequence of the nucleic acid template. For example, in the step S150, the weighting scheme may be related to an estimated probability that the unmutated sequence read was derived from the same copy of a repeat region (such as the same haplotype and/or location in a genome) as a mutated sequence read to which the unmutated sequence read is aligned. For example, in some embodiments, each pairwise alignment of an unmutated sequence read to a mutated sequence read has one weight based on an estimated probability that the unmutated sequence read and the mutated sequence read are derived from the same copy of the repeat region. Such estimated probability may be iteratively updated as described above. In the step S150, the weighting scheme may also be related to a current iterative estimate of the most probable sequence of the nucleic acid template, where the most probable sequence is iteratively updated as described herein. Thus, in some embodiments, in step S150, the weighting scheme may iteratively update weighted contributions of each unmutated sequence read aligned to one or more mutated sequence reads at a given site, based on current estimated probabilities of sequence identity of the unmutated sequence read, and based on a current predicted most probable sequence of the nucleic acid template.

In the step S150, contributions of unmutated sequence reads may be weighted based on the current estimated probabilities, such as the estimated probabilities based on the current iteration of the optimization process. Thus, the estimated probabilities for the unmutated sequence reads can then be factored in when determining a most probable sequence. In the step S160, the process proceeds to updating an inference of a most probable base for the site. In some embodiments, the inference of a most probable base includes a calculation of a probability distribution over bases (such as over A,C,G,T) for the site. In some embodiments, the most probable base is a base which has the highest statistical likelihood of matching the nucleic acid template at the site of the base. In some embodiments, in step S160, the method may incorporate the weighted contributions of the unmutated sequence reads in updating the inference of a most probable base for the site. In some embodiments, in step S160, the inference of a most probable base is updated for each site in a subset of sites.

In some embodiments, in step S140, updated probabilities can be re-estimated using the updated inference of a most probable base of the step S160. In the step S150, the updated estimated probabilities may be used to weight contributions of the unmutated sequence reads, and to, in the step S160, update an inference of a most probable base, such as for a second site or subset of sites. In some embodiments, the process is iterated until the change to the resulting most probable sequence or probability distribution over bases is below a convergence threshold.

An advantage of using such an optimization process as described in the step S130 is that the optimization process iteratively progresses towards a specific copy of a repeat region of a nucleic acid template in a nucleic acid template sample, rather than determining an average sequence across multiple copies. By progressing toward a specific copy of a repeat region, the method allows for the determination of the most probable sequence of a particular copy of a repeat region in a nucleic acid template, rather than producing an “average” of the repeat regions across the different copies of the repeat region in the nucleic acid sample. Thus, in some embodiments, methods and systems described herein include determining a sequence of a copy (such as one copy out of two or more non-identical copies) of a repeat region. In some embodiments, where the two or more copies of a repeat region in the nucleic acid sample include two or more haplotypes, the method includes determining a sequence of a haplotype (such as one haplotype out of two or more non-identical haplotypes) of the nucleic acid sample.

By way of illustrative example, in some embodiments, the optimization process of the step S130 includes applying an Expectation Maximation method. An Expectation Maximization (EM) is a statistical inference method that is commonly employed when there are dependencies among parameters in a probabilistic model that prevent the direct use of analytical techniques for calculating expectations, means, variances, etc. By way of nonlimiting example, under an EM iterative approach, in step S130, an alignment of mutated sequence reads to unmutated sequence reads is taken as the input. Sites may be evaluated on a site-by-site basis to find sites with high confidence as to the most probable base for the site. In the step S160, an inference of a most probable base may be updated by calculating a probability distribution over the bases (such as over A,C,G,T) for the site. Then, the probability distribution over the bases (or the collection of such distributions among all aligned sites) may be used to re-estimate the probabilities of unmutated sequence read alignments in step S140. The updated alignments can be re-evaluated for areas of high confidence. Sites now with sufficiently high confidence can then be used to determine a most probable base for the site. The process can be repeated until a convergence threshold has been reached.

In some embodiments, in step S130, the optimization process incorporates information that describes variations in read length. A problem may arise in real (non-simulated) datasets, where sequence reads may be unequal length due to properties of the sequencing chemistry and preliminary data analysis steps such as read trimming, and information about read length may generally factor into an optimization process. Thus, information about such variation in sequence read length may be incorporated in the optimization process. For example, in some embodiments, the EM method uses a pseudolikelihood defined on a fixed data dimension (read length).

### Summarizing Unmutated Sequence Reads and Incorporating Information

In some embodiments, a process of calculating an inference of a most probable base includes a step of summarizing the probability of each base at a site associated with the unmutated sequence reads. For example, in some embodiments, step S160 of updating an inference of a most probable base includes a step S161 of, on a site-by-site basis, summarizing the probability of each base at each site associated with the unmutated sequence reads. For example, in some embodiments, for each site associated with the unmutated sequence reads, a probability that nucleotides A, C, G, or T is at a particular position in the unmutated sequence reads is summarized. In some embodiments, summarizing may be expressed as equation (3) in Example 1.

In some embodiments, a process of calculating an inference of a most probable base includes a step of incorporating information related to a probability of a mutation at a site, related to a probability of an error (such as unintentionally introduced mutations or such as sequencing error), and/or related to variation in mutation rates and local sequence composition. For example, following the site-by-site analysis at step S161, the process may include a step S162 of incorporating information related to the probability of a mutation at each site of a mutated sequence read. For example, in the case of intentionally introduced mutations, the probability of a mutation at each site in the mutated sequence reads may be statistically inferred based on knowledge about the mutations rates of the mutagenesis process used in the sample preparation formulation.

In some embodiments, the step S162 includes incorporating information that describes a probability of an error associated with a sample preparation or sequencing process. For example, in some embodiments, in the step S162, the method includes accounting for changes other than intentionally introduced mutations (such as unintentionally introduced mutations). In some embodiments, in the step S162, the method accounts for changes in the sequence such as those introduced by the use of PCR during sample preparation and sequencing, or other sources of sequencing error. In some embodiments, in the step S162, such other changes may be accounted for in combination with the probability of an intentionally-introduced mutation at a site.

Depending on the mutagenesis process, sample preparation, library preparation, and sequencing methods used, the probability of an intentionally-introduced mutation may differ from the probability of an unintentional change to the sequence in the mutated sequence reads. For example, in the step S162, the method may account for at least four different possible scenarios having occurred at each site in the mutated sequence read: 1) an intentional mutation (such as a transition mutation) and no unintentional mutation (such as polymerase error); 2) an unintentional mutation and no intentional mutation; 3) both intentional mutation and unintentional mutation; and 4) neither intentional mutation nor an unintentional mutation.

In some embodiments, this process includes determining a most probable base for a site of a mutated sequence read that is not aligned to an unmutated sequence read, based on a statistically inferred probability of a mutation at the site. For example, in some embodiments, some sites in a mutated sequence read may not align (may not map) to any unmutated sequence read. In some embodiments, in the absence of information about the site from the unmutated sequence reads, the most probable base for the site may be determined based only on the mutated sequence read(s) covering the site, combined with the statistically inferred probability that a mutation occurred at the site. In some embodiments, the per-base accuracy score for the site would also be based on the same process.

In some embodiments, in the step S162, this process includes incorporating information that describes variation in mutation rate between mutated sequence reads or unequal mutation rates of adenine, cytosine, guanosine, and thymine. In some embodiments, in the step S162, the process may include incorporating information that describes variations in mutation rates between mutated sequence reads, when statistically predicting a most probable sequence. For example, while a mutagenesis process may have an average mutation rate, for example 5%, some mutated sequence reads may randomly have more or less mutations than average, such as 3% or 8%. Similarly, in the step S162, this process may include incorporating information that describes unequal mutation rates of adenine, cytosine, guanosine, and thymine, when statistically predicting a most probable sequence. For example, a mutagenesis process may produce varied mutation rates among A, C, G, T nucleotide sites, which information may be incorporated into the methods described herein.

### Determining a Most Probable Sequence and Per-Base Accuracy Score

In some embodiments, after the optimization process at the step S130, the method further includes a step S170 of determining: i) a most probable sequence of the nucleic acid template, and ii) for each base of the most probable sequence, a per-base accuracy score correlated with the probability that the base matches with the nucleic acid template.

In some embodiments, the per-base accuracy score includes a per-base Quality Value (QV), also known as a “PHRED” score. In some embodiments, the per-base accuracy score estimates the statistical confidence in the prediction of each base. In some embodiments, the method further comprises determining an overall accuracy score related to the most probable sequence. In some embodiments, the overall accuracy score is determined based on the per-base accuracy scores of each of the bases in the most probable sequence. For example, the overall accuracy score may be an average or median of the per-base accuracy scores, or another composite of the per-base accuracy scores.

In some embodiments, the step S170 of determining a most probable sequence of the nucleic acid template includes determining a most probable base for each site associated with the alignment of the unmutated sequence reads to the mutated sequence reads as described herein. In some embodiments, the method includes determining a most probable sequence proceeds on a site-by-site basis. For example, the method may include statistically inferring a most probable base for one site (position in a nucleic acid sequence) by analyzing the alignment of the mutated sequence reads to the unmutated sequence reads at that site, then proceeding to another site, and so on. For example, in some embodiments, the method includes determining a maximum a posteriori base for each site, and the most probable sequence includes one or more maximum a posteriori bases. In some embodiments, determining a most probable base for each site includes calculating an inference of a most probable base for the site, such as by calculating a probability distribution over bases (such as A,C,G,T) at the site. In some embodiments, the most probable sequence includes one or more most probable bases determined by, for example, applying an optimization process in the step S130.

In some embodiments, the step S170 of determining a most probable sequence of the nucleic acid template includes determining a most probable sequence of a subset of sites associated with the alignment of the unmutated sequence reads to the mutated sequence reads. Thus, in some embodiments, the method includes determining a most probable sequence proceeds on a subset-by-subset basis. As used herein, a subset of sites refers to a group of sites included within a most probable sequence. In some embodiments, the subset of sites is smaller than the total number of sites within a most probable sequence.

In some embodiments, the most probable sequence and per-base accuracy score(s) are stored in a file on a computer storage medium (such as a computer hard drive, for example a spinning magnetic disk drive or a solid state drive). In some embodiments, the file is a stored in the format of a BAM, SAM, CRAM, or VCF file.

### Selecting a Subset of Unmutated Sequence Reads

In some embodiments, the systems and methods of the present invention include methods of selecting a subset of unmutated sequence reads among unmutated sequence reads derived from different copies of a repeat region. In some embodiments, selecting a subset of unmutated sequence reads includes selecting a subset of unmutated sequence reads, which are aligned to one or more mutated sequence reads at a site, to contribute to the statistical inference of a most probable base of the nucleic acid template at that site. In some embodiments, other non-selected unmutated sequence reads aligned to the mutated sequence reads at the site are excluded from contributing or contribute less to the statistical inference of a most probable base of the nucleic acid template at that site. In some embodiments, after the most probable base for the site is statistically inferred as described herein, the process of selecting a subset of unmutated sequenced reads is iterated at a second site. In some embodiments, the subset of unmutated sequences is selected among unmutated sequence reads derived from different copies of repeat regions in the nucleic acid sample.

In some embodiments, selecting a subset of unmutated sequence reads comprises scoring alignments of unmutated sequence reads to the mutated sequence reads and selecting top-scoring alignments. In some embodiments, the scoring scheme is specific to the alignment of the unmutated sequence reads to the mutated sequence reads at a particular site, such as the site for which a most probable base is predicted. For example, in some embodiments, alignments of unmutated sequence reads to the mutated sequence reads are scored using an alignment scoring scheme and selecting the best “n” number of alignments under the alignment scoring scheme.

Various scoring schemes may be used to score the alignments in embodiments of the invention. By way of nonlimiting example, in some embodiments, a standard scoring scheme is used to score the alignments. In some embodiments, the scoring scheme may be a 4×4 substitution matrix and affine gap penalty score. In some embodiments, the scoring scheme is a probability generated by integrating alignments using a method such as a pair-Hidden Markov Model (pair-HMM). In some embodiments, another method known in the art is used to score the alignments of unmutated sequence reads to the mutated sequence read.

By way of nonlimiting example, after scoring the alignments, an iterative process is applied to select the top-scoring of alignments for each site. For example, for a particular site, the top-scoring “n” number of alignments for that particular site are selected, where “n” refers to a determined number of alignments. For example, “n” may be a number such as 20, such that the top 20 alignments are selected. “n” may be 2, 5, 10, 15, 20, 25, 30, 40, 50, or a range constructed from any of the aforementioned values. In some embodiments, “n” is related to the depth of coverage of unmutated sequence reads used in an alignment. For example, in some embodiments, “n” corresponds to 50% of the depth of coverage, where if coverage is 40×, “n” is 20.

In some embodiments, after the top-scoring alignments are selected for the site, a most probable base (such as a maximum a posteriori base) is determined as described herein. This most probable base may be incorporated into the most probable sequence. In some embodiments, the process is iterated for additional sites using the current estimate of the most probable sequence. For example, the process may be iterated until fewer than a specified fraction of the bases change as a result of the iteration. A per-base accuracy score may be determined for each a base and further described above. In some embodiments, a most probable sequence and a per-base accuracy score is thus determined.

### Systems

In another aspect, disclosed herein are systems. For example, a system may comprise one or more processors configured to perform any method described herein that is capable of computer implementation. In some embodiments, disclosed herein is a system comprising a processor configured to perform a method comprising: a) aligning unmutated sequence reads of a nucleic acid template to mutated sequence reads, wherein the mutated sequence reads comprise one or more mutations, and b) determining: i) a most probable sequence of the nucleic acid template, and ii) for each base of the most probable sequence, a per-base accuracy score correlated with the probability that the base matches with the nucleic acid template; thereby determining the sequence of the nucleic acid template.

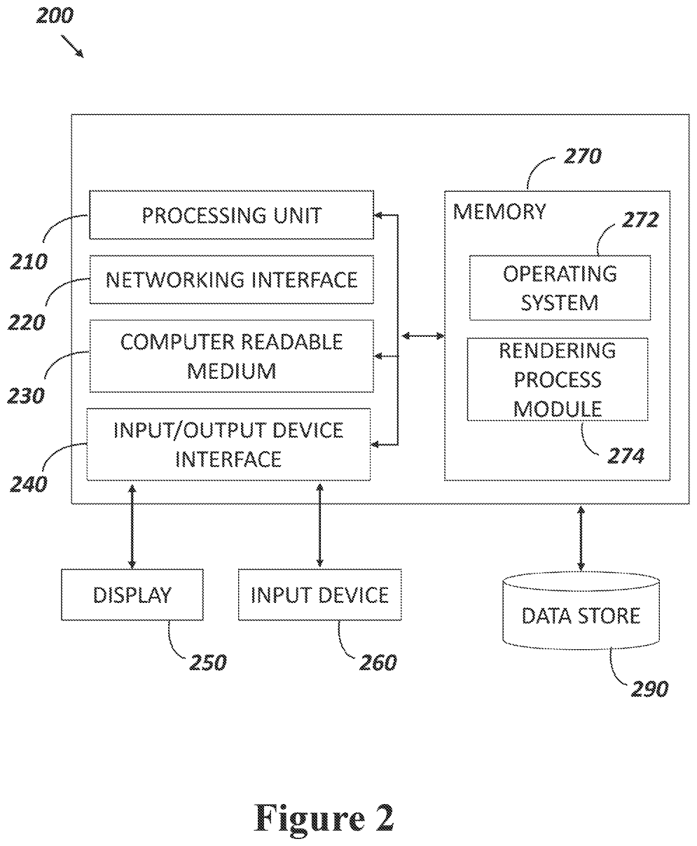

FIG. 2 is a block diagram of an exemplary computing device 200 that may be used in connection with an illustrative sequencing system. The computing device 200 may be configured to determine a sequence of a nucleic acid template by removing mutations found in a mutated sequence read. The general architecture of the computing device 200 depicted in FIG. 2 includes an arrangement of computer hardware and software components. The computing device 200 may include many more (or fewer) elements than those shown in FIG. 2. It is not necessary, however, that all of these generally conventional elements be shown in order to provide an enabling disclosure.

As illustrated, the computing device 200 includes a processing unit 210, a network interface 220, a computer readable medium drive 230, an input/output device interface 240, a display 250, and an input device 260, all of which may communicate with one another by way of a communication bus. The network interface 220 may provide connectivity to one or more networks or computing systems. The processing unit 210 may thus receive information and instructions from other computing systems or services via a network. The processing unit 210 may also communicate to and from memory 270 and further provide output information for an optional display 250 via the input/output device interface 240. The input/output device interface 240 may also accept input from the optional input device 260, such as a keyboard, mouse, digital pen, microphone, touch screen, gesture recognition system, voice recognition system, gamepad, accelerometer, gyroscope, or other input device.

The memory 270 may contain computer program instructions (grouped as modules or components in some embodiments) that the processing unit 210 executes in order to implement one or more embodiments. The memory 270 generally includes RAM, ROM and/or other persistent, auxiliary or non-transitory computer-readable media. The memory 270 may store an operating system 272 that provides computer program instructions for use by the processing unit 210 in the general administration and operation of the computing device 200. The memory 270 may further include computer program instructions and other information for implementing aspects of the present disclosure.

For example, in one embodiment, the memory 270 includes a rendering process module 274 for determining a sequence of a nucleic acid template by removing mutations found in a mutated sequence read. The rendering process module 274 can perform the methods disclosed herein, including the process described with respect to FIG. 1. In addition, memory 270 may include or communicate with the data store 290 and/or one or more other data stores that store one or more inputs, one or more outputs, and/or one or more results (including intermediate results) of determining a sequence of a nucleic acid template by removing mutations found in a mutated sequence read according to the present disclosure. Such data may include, for example, unmutated sequence reads, mutated sequence reads, mutation probability information, error probability information, variable mutation rate information, and the most probable sequence and per-base accuracy scores.

In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD-ROM, DVD-ROM, memory stick, flash drive, hard drive, SSD hard drive, server, mainframe storage system and the like.

In some embodiments, the methods may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being Produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (for example, iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework. (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims. For example, the notation introduced in some of the examples below assumes alignment of unmutated sequence reads and mutated sequence reads to a reference genome assembly. However, the methods described below may also be used in direct alignments of unmutated sequence reads to mutated sequence reads, without the use of a reference sequence as an intermediate.

In the examples below, the following notation is applicable. “Dream” or “dream sequence” refer to mutated sequence reads, which contain mutations that may be removed by rendering.

Denote the set of mapped “ref reads” (unmutated sequence reads, such as standard unmutated shotgun reads, from a reference sequence) that cover site i in the reference sequence as Rᵢ and let r∈Rᵢ be a read from the set of reads that align to site i in the reference sequence. Denote the nucleotide vector in read r where it has aligned to site i in the reference sequence as rᵢ. rᵢ is a length 4 indicator vector corresponding to one of the four nucleotides (for example, for A is [1; 0; 0; 0] C is [0; 1; 0; 0]). Denote the probability that the basecall at site i in read r is correct, for example, the quality score, as q(rᵢ).

Let dᵢ denote a vector of length 4 describing the probability of A, C, G, and T at the position in a dream aligned to reference site i, in terms of a length 4 indicator vector aᵢ with a 1 in the position corresponding to A,C,G,T of the dream base. As above, let q(aᵢ) denote the probability that the basecall at site i of the dream sequence matches with the nucleic acid template. Dᵢ is written as follows:

$${d}_{i} = {a}_{i} q ( {a}_{i} ) + ( 1 - {a}_{i} ) ( 1 - q ( {a}_{i} ) / 3 ) \qquad ( 1 )$$

### Example 1

Statistical methods were used to determine a sequence of a nucleic acid by removing mutations introduced into a mutated sequence read. The details of the statistical methods are described below.

### Maximum a Posteriori Basecalling and Bayes Factor Quality Scores

Let:

$${x}_{i} = [ α , α , α , α ] + {\sum }_{{r}_{i} ∈ {R}_{i}} q ( {r}_{i} ) {r}_{i} + ( 1 - {r}_{i} ) ( 1 - q ( {r}_{i} ) ) / 3 \qquad ( 2 )$$

the vector xᵢ is an unnormalized distribution over the ref read bases at site i, and is a regularization prior/pseudocount used to force all base probabilities to be equal in the event that there is no reliable data from the mapped ref reads. When normalized, this forms a probability distribution over A,C,G,T at site i:

$${y}_{i} = \frac{{x}_{i}}{\sum {x}_{i}} \qquad ( 3 )$$

Denote the prior probability of mutation at any site as P(u) where u refers to a mutation event. For example, in a typical application of Illumina® Complete Long Read (ICLR) sequencing, P(u) would take on values in the range of 0.05 to 0.06.

Let zᵢ denote a length 4 vector of the unnormalized posterior probabilities of A,C,G,T at site i. The values of zᵢ are defined as follows:

$${z}_{i} = P ( u ) ( {d}_{i} M ) ∘ {y}_{i} + ( 1 - P ( u ) ) ( {d}_{i} ∘ {y}_{i} ) \qquad ( 4 )$$

Where ∘ denotes the Hadamard product of vectors/matrices (the elementwise product) and where yᵢ is a normalized vector of length 4 describing the probability of A, C, G, and T at that position in the ref sequence, the computation of which from ref reads is described as above. M is a 4×4 rotation matrix that rotates the elements of a length 4 vector to apply a transition mutation:

$$M = [ \begin{matrix}0 & 0 & 1 & 0 \\ 0 & 0 & 0 & 1 \\ 1 & 0 & 0 & 0 \\ 0 & 1 & 0 & 0\end{matrix} ] \qquad ( 5 )$$

To render a base, a maximum a posteriori value is selected from zᵢ:

$${d}_{i}^{′} = arg max ( {z}_{i} ) \qquad ( 6 )$$

where d′ᵢ denotes the rendered dream base (most probable base) at alignment site i.

The quality score is set for site i, denoted as QV(zᵢ), equal to:

$$QV ( {z}_{i} ) - 10 {log}_{10} \frac{- max ( {z}_{i} ) + \sum {z}_{i}}{max ( {z}_{i} )} \qquad ( 7 )$$

The quality score is a Bayes factor comparing the hypothesis that the base is not argmax(zᵢ), the numerator, to the hypothesis that the base is argmax(zᵢ) (denominator).

### Example 2: Extension of MAP Basecalling to Polymerase Error Correction

The basecalling methods described above were extended to determine a sequence when accounting for changes other than transition mutations, such as those introduced by the use of PCR during sample preparation and sequencing, or other sources of sequencing error. The details of this method are described below.

To account for non-transition polymerase error, let

$$E = [ \begin{matrix}0 & 1 & 1 & 1 \\ 1 & 0 & 1 & 1 \\ 1 & 1 & 0 & 1 \\ 1 & 1 & 1 & 0\end{matrix} ] \qquad ( 8 )$$

And let PI be the prior probability of a polymerase error, for example, expected to be on the order of 5×10⁻⁵ per cycle of PCR or 1.6×10⁻³ in a typical ICLR library (32 total cycles). The posterior probabilities over bases are then redefined as:

$${z}_{i} = ( 1 - P ( e ) ) P ( u ) ( {d}_{i} M ) ∘ {y}_{i} + P ( e ) ( 1 - P ( u ) ) ( {d}_{i} E ) ∘ {y}_{i} + P ( e ) P ( u ) ( {d}_{i} ME ) ∘ {y}_{i} + ( 1 - P ( e ) ) ( 1 - P ( u ) ) ( {d}_{i} ∘ {y}_{i} ) \qquad ( 9 )$$

Intuitively, the equation above integrates probabilities over four scenarios, reflected in the four terms of the equation. The first term corresponds to a mutation event and no polymerase error, the second term a polymerase error and no mutation, third term both mutation and polymerase error, and the fourth term when neither mutation nor error have occurred. Basecalling and QV estimation proceeds in the same manner as described above in equations (6) and (7), but using the revised definitions of zᵢ. That is, the output base is set at position i to the nucleotide corresponding to argmax(z) and the quality score QV(zᵢ) is computed as a Bayes factor comparing the MAP base to the other three candidate bases.

### Example 3: Handling of Unmapped Dream Positions

A statistical method was used to determine a sequence when a site in a mutated sequence read are unmapped to a unmutated sequence read. The details of the statistical method are described below.

As above, let zᵢ denote a length 4 vector of the posterior probabilities of bases at site i. Define ϕ as a length four vector representing the probability that any A,C,G,T base in any dream has been mutated. ϕ extends the concept of P(u) to represent unequal probability of mutation at sites that contain A,C,G,T. This quantity is calculated reliably by simply counting the number of transition differences at all A,C,G,T sites in the alignment of the set of all dreams to the ref reads and normalizing by the total number of A,C,G,T sites. Formally, ϕ is defined as:

$$ϕ = [ \frac{{\sum }_{𝒜 : {a}_{i} = A ⋀ {r}_{i} = G}}{{\sum }_{𝒜 : {a}_{i} = A}} , \frac{{\sum }_{𝒜 : {a}_{i} = C ⋀ {r}_{i} = T}}{{\sum }_{𝒜 : {a}_{i} = C}} , \frac{{\sum }_{𝒜 : {a}_{i} = G ⋀ {r}_{i} = A}}{{\sum }_{𝒜 : {a}_{i} = G}} , \frac{{\sum }_{𝒜 : {a}_{i} = T ⋀ {r}_{i} = C}}{{\sum }_{𝒜 : {a}_{i} = T}} ] \qquad ( 10 )$$

where aᵢ and rᵢ are the indicator vectors for dreams and reads defined above and A, C, G, Tare shorthand for indicator vectors corresponding to each base. As such, ϕ is defined as a length 4 vector, with one term for each of A,C,G,T giving the rate at which transition differences are observed between the aligned positions aᵢ in the dreams and ref reads rᵢ in the set of alignments [symbol] . ϕ is estimated using a collection of one or more aligned dreams, and the resulting estimate of ϕ is then be applied when rendering unaligned dreams. That is, the values of ϕ are expected to be similar among dreams that are aligned and those that are unaligned in a particular sample/dataset, because they were all subjected to the same mutation process during sample prep.

If the dream base is unaligned, then let

$${z}_{i} = {d}_{i} ∘ ( 1 - ϕ ) + ( 1 - {d}_{i} ) ∘ ϕ \qquad ( 11 )$$

where i is reinterpreted as an index into the dream sequence rather than a sequence alignment index. The first term integrates the likelihoods in the case where no mutation has occurred, and the second term handles the case where a mutation has occurred. As above, the output base at site i is set to argmax(z) using equation (6) and the QV is set using equation (7). In this case, because there is no information from ref reads, the maximum a posteriori (MAP) estimate of the base and the associated quality score is driven entirely by data about the overall mutation rate.

### Example 4: Adjustment for Local Sequence Composition and Unequal Mutation Rates

A statistical method was used to determine a sequence when accounting for local sequence composition and unequal mutation rates. The details of the statistical method are described below.

Variation in local sequence composition and unequal mutation rates at the four bases A,C,G,T is accounted for by use of ϕ, as defined above in section (see equation (10)). To do so, the definition of z; is further extended as follows:

$${z}_{i} = ( 1 - P ( e ) ) ϕ ∘ {d}_{i} M ∘ {r}_{i} + P ( e ) ( 1 - ϕ ) ∘ {d}_{i} E ∘ {r}_{i} + P ( e ) ϕ ∘ {d}_{i} ME ∘ {r}_{i} + ( 1 - P ( e ) ) ( 1 - ϕ ) ∘ {d}_{i} ∘ {r}_{i} \qquad ( 12 )$$

### Example 5: Expectation Maximization

An EM-like technique was applied to infer a rendered sequence. The details of the technique are described below.

Define the likelihood [symbol] (r|Θ,ι) of read r, present in an alignment of [symbol] of r to the dream using the following:

$$ℒ ( r \vert Θ , ι ) = {\prod }_{i ∈ {𝒜}_{r}} Z ( e , {ϕ}_{ι} , {d}_{i , ι} , {r}_{i} )$$

where Θ takes on the usual meaning as notation for an aggregate of all the model parameters; where rᵢ is the length four probability vector of A,C,G,T in read r at site i in the alignment of the read to the dream, and ι={0, 1, 2} denotes the values produced by an iteration of EM. Z(⋅) is simply the previously introduced equation (12) recast as a function with parameters that can change across iterations of EM:

$$Z ( e , ϕ , {d}_{i , ι} , {r}_{i} ) = ( 1 - P ( e ) ) ϕ ∘ {d}_{i} M ∘ {r}_{i} + P ( e ) ( 1 - ϕ ) ∘ {d}_{i} E ∘ {r}_{i} + P ( e ) ϕ ∘ {d}_{i} ME ∘ {r}_{i} + ( 1 - P ( e ) ) ( 1 - ϕ ) ∘ {d}_{i} ∘ {r}_{i} \qquad ( 14 )$$

The remaining notation is as previously defined. The values of ϕ₀ are initialized as described above in Equation (10). The values of d_{i,0} are initialized as above for dᵢ in equation (1).

The parameter updates for each iteration of EM are then defined as follows. First, x and y are redefined to use the per-read likelihoods, and to be indexed by EM iteration:

$${x}_{i , ι} = [ α , α , α , α ] + {\sum }_{r ∈ R} ℒ ( r \vert Θ , ι ) ( q ( {r}_{i} ) {r}_{i} + ( 1 - {r}_{i} ) ( 1 - q ( {r}_{i} ) ) / 3 ) \qquad ( 15 )$$

$${y}_{i , ι} = \frac{{x}_{i , ι}}{\sum {x}_{i , ι}} \qquad ( 16 )$$

The c vector sums the likelihoods of reads used at each site, and is used in the definition of y_{i,ι} to normalize the values to sum to 1. As above, α is a regularization constant.

The previously defined Z(⋅) is then used to identify the maximum likelihood base (most probable base) at each site, which is denoted:

$${d}_{i , ι}^{′} = arg max Z ( e , {ϕ}_{ι} , {d}_{i , ι} , {y}_{i , ι} ) \qquad ( 17 )$$

And let:

$${d}_{i , ι} = \frac{Z ( e , {ϕ}_{ι} , {d}_{i , ι} , {y}_{i , ι} )}{\sum Z ( e , {ϕ}_{ι} , {d}_{i , ι} , {y}_{i , ι} )} \qquad ( 18 )$$

As above in equation (6)′ d′ is used to denote the bases in a fully resolved nucleotide sequence. d remains as the probability distribution over the four possible nucleotides at each site, updated at each iteration of EM. All that remains is to define the values of ϕ at each iteration, which can be done in terms of d_{i,ι}′:

$${ϕ}_{i} = [ \frac{{\sum }_{𝒜 : {d}_{i , ι}^{′} = A ⋀ {r}_{i} = G}}{{\sum }_{𝒜 : {d}_{i , ι}^{′} = A}} , \frac{{\sum }_{𝒜 : {d}_{i , ι}^{′} = C ⋀ {r}_{i} = T}}{{\sum }_{𝒜 : {d}_{i , ι}^{′} = C}} , \frac{{\sum }_{𝒜 : {d}_{i , ι}^{′} = G ⋀ {r}_{i} = A}}{{\sum }_{𝒜 : {d}_{i , ι}^{′} = G}} , \frac{{\sum }_{𝒜 : {d}_{i , ι}^{′} = T ⋀ {r}_{i} = C}}{{\sum }_{𝒜 : {d}_{i , ι}^{′} = T}} ]$$

That is, ϕ_{ι} is defined as a length 4 vector, with one term for each of A,C,G,T in d_{i,ι}′, giving the rate at which transition differences are observed between the aligned positions in d_{i,ι}′, and ref reads in the set of alignments [symbol] .

### Example 6: Practical Considerations with Real Datasets

A statistical method was used to adjust the expectation maximization method described above, which assumes reads of equal length so that the likelihoods are computed on data of equal dimension, to account for reads of unequal length. The details of the statistical method are described below.

Denote L as the maximum read length that will be processed. In an exemplary application, L=150. Alternatively, L=300 is taken if paired-end 150 nt (150-nucleotide) reads are being processed as a single entity. For a particular read, I( [symbol] ) as the number of sites in read r that were aligned to the dream, and define:

$${q}_{r} = \frac{{\sum }_{i ∈ {𝒜}_{r}} Z ( e , {ϕ}_{ι} , {d}_{i , ι} , {r}_{i} )}{l ( {𝒜}_{r} )}$$

That is, qᵣ is the average per-site likelihood for the aligned sites in the read. qᵣ can be used to define a pseudolikelihood of fixed-dimension for read r, denoted as [symbol] *:

$${ℒ}^{*} ( r \vert Θ , ι ) = {q}_{}^{L - l ( {𝒜}_{r} 0} {\prod }_{i ∈ {𝒜}_{r}} Z ( e , {ϕ}_{l} , {d}_{i , ι} , {r}_{i} ) \qquad ( 20 )$$

In other words, the average per-site likelihood from aligned sites is taken as an estimate of what the per-site likelihood would be at any missing sites, to pad the data dimension in the likelihood function out to L sites.

Finally, a function Δ(⋅) is defined that counts the number of nucleotide differences between two sequences as follows:

$$Δ ( {d}_{\cdot , ι - 1}^{′} , {d}_{ , ι}^{′} ) = {\sum }_{i} \{ \begin{matrix}0 & if {d}_{i , ι - 1}^{′} = {d}_{i , ι}^{′} \\ 1 & if {d}_{i , ι - 1}^{′} \neq {d}_{i , ι}^{′}\end{matrix} \qquad ( 21 )$$

An exemplary method can be expressed in terms of the defined notation and functions as:

EM Method 1: Expectation maximization rendering

| | |
|---|---|
| 1: procedure RenderEM(a, b) | [symbol] Method to render via EM |
| 2: Initialize ϕ₀ using Equation (10) | |

3: d_{i,0} ← aᵢq(aᵢ) + (1 − aᵢ)(1 − q(aᵢ)/3) [symbol] initial probabilities of bases in the

| | |
|---|---|
| mutated sequence | |
| 4: d′_{i,0} ← aᵢ | [symbol] The initial mutated sequence |
| 5: ι ← 1 | |

6: δ_{ι} ← ∞ [symbol] Counts the number of sequence changes introduced

by the previous iteration
7: while δ_{ι} ≠ 0 do

8: x_{i,ι} ← [α, α, α, α] + Σ_{r∈R} [symbol] (r|Θ, ι − 1)(q(rᵢ) rᵢ + (1 − rᵢ) (1 − q(rᵢ))/3) [symbol] Equation (20)

| | |
|---|---|
| $9 : {y}_{i , ι} ← \frac{{x}_{i , ι}}{\sum {x}_{i , ι}}$ | [symbol] Equation (16) |
| $10 : {d}_{i , ι} = \frac{Z ( e , {ϕ}_{ι - 1} , {d}_{i , ι - 1} , {y}_{i , ι} )}{\sum Z ( e , {ϕ}_{ι - 1} , {d}_{i , ι - 1} , {y}_{i , ι} )}$ | [symbol] Equation (18) |
| 11: d′_{i,ι} = argmax Z(e, ϕ_{ι-1}, d_{i,ι-1}, y_{i,ι}) | [symbol] Equation (17) |

12: Compute ϕ_{ι} as described in Equation (19)

| | |
|---|---|
| 13: ι ← ι + 1 | |
| 14: δ_{ι} ← Δ(d′ [symbol] _{ι-1},d′ [symbol] ) | [symbol] Equation (21) |
| 15: end while | |
| 16: return d′ [symbol] _{ι-1} | [symbol] The rendered sequence |
| 17: end procedure | |

[symbol]

indicates data missing or illegible when filed

### Example 7: Mutation in Simple Simulated Data Set

A statistical method as described herein was used to render a mutation in a simulated data set with no sequencing error.

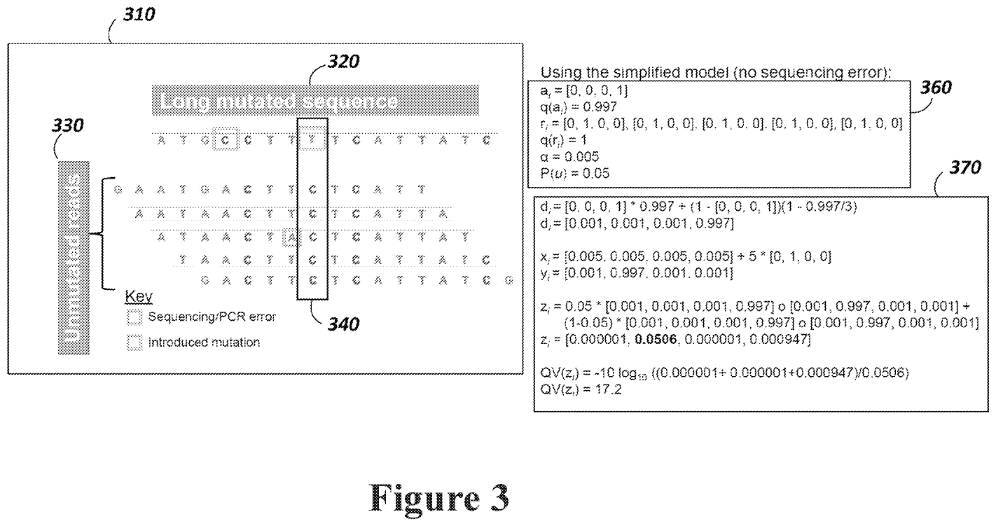

For example, FIG. 3 depicts an example of rendering a site 340 that contains a mutation. In alignment 310, a mutated sequence read 320 is compared to several aligned unmutated sequence reads 330, and a mismatch is highlighted at site 340 where a mutated sequence read has “T” at the site 340, while the unmutated sequence reads have a “C” at the site 340. The simplest version of the mathematical model of the mutation and sequencing process is used in the example of FIG. 3. In this example, the unmutated sequence reads 330 were unanimous in their support for an alternate nucleotide compared to the mutated sequence read 320, and the difference was explained by a transition mutation. The maximum a posteriori base produced by the model matched the base present in the unmutated sequence reads 330, so the rendering process corrected the mutation. In the example of FIG. 3, the simple model outlined in equations (6) and (7) was used. The alignment of unmutated sequence reads 330 to a mutated sequence read 320 is shown in alignment 310. Values for aᵢ, q(aᵢ), rᵢ, q(rᵢ), αᵢ and P(u) are specified as constants in table 360 The use of the constant values in the equations to compute dᵢ, xᵢ, yᵢ, zᵢ and QV(zᵢ) is shown in table 370. The MAP base selected from zᵢ is C (corresponding to the maximum value in zᵢ of 0.0506), and the resulting PHRED-scaled QV score is 17.2, corresponding to an estimated probability of about 0.98 that the rendered base call is correct. In a typical dataset with higher coverage of unmutated data (for example, a greater number of aligned unmutated sequence reads 330), the QV score would be higher.

### Example 8: SNP in Simple Simulated Data Set

A statistical method as described herein was used to render a mutation in a simulated data set with no sequencing error. The data set of this example contained at least two haplotypes that are heterozygous for a single nucleotide polymorphism (SNP).

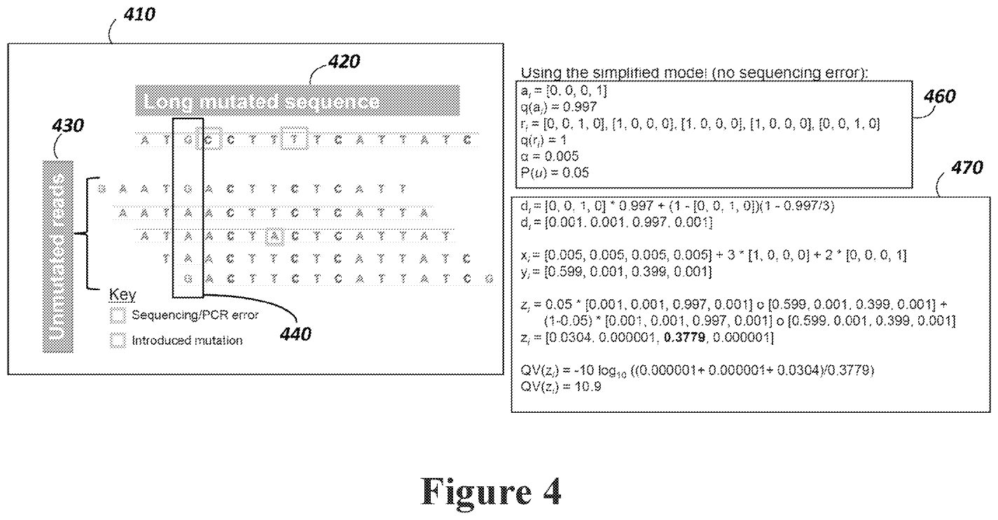

FIG. 4 depicts an example of rendering a site where the unmutated data shows a heterozygous SNP, where the two bases involved represent a transition difference. In alignment 410, the mutated sequence read 420 matches one of the two bases present at site 440 in the unmutated sequence reads 430, which leaves a high degree of uncertainty as to whether or not the site 440 was mutated. In table 470, the MAP estimate produced by the model leaves the base unchanged, and assigns it a low QV, corresponding to a probability of about 0.92 that the base has been called correctly. The low confidence in the basecall reflects the a priori knowledge that about 5% of sites were mutated in the sequence, due to the nature of the sequencing chemistry. In the example of FIG. 4, the simple model outlined in equations (6) and (7) was used. The alignment of unmutated sequence reads to a mutated sequence read is shown in alignment 410. Values for dᵢ, q(aᵢ), rᵢ, q(rᵢ), a; and P(u) are specified as constants in table 460. The use of the constant values in the equations to compute dᵢ, xᵢ, yᵢ, zᵢ and QV(zᵢ) is shown in table 470. The MAP base selected from zᵢ is G (corresponding to the maximum value in zᵢ of 0.3779), and the resulting PHRED-scaled QV score is 10.9, corresponding to an estimated probability of about 0.92 that the rendered base call is correct.

### Example 9: Accuracy Evaluation in Simulated Datasets

The accuracy of rendering methods was evaluated on a simulated dataset which included mutated sequence read templates, a “reference genome” from which the mutated templates were simulated, and unmutated “reference reads” (unmutated sequence reads), also simulated from the reference genome. An advantage of evaluation on simulated datasets was that the correct rendered sequence (corresponding to the sequence of the nucleic acid template) was known, and so the accuracy of rendering was evaluated unambiguously. The details of the experiment are described below.

### Generation of Simulated Data

Input to the simulation process included a reference genome assembly, the target coverage for ref reads, the target coverage for long reads, and the short read coverage per long read template (redundancy).

The simulation process produced several outputs, including the true set of long template sequences (representing the nucleic acid template sequence), and their locations in the reference genome assembly, the set of long template sequences with simulated mutations (representing the mutated nucleic acid template), the locations of the mutations that were introduced to the long templates, a set of standard short reads (representing unmutated sequence reads), a set of short reads simulated from the mutated long template sequences (representing mutated sequence reads), and information on which long template each mutated short read originated from.

The simulation system was used to generate three datasets, referred to as “small,” “medium,” and “large,” with the characteristics listed in Table 1 below. The human simulation used a diploid assembly of HG002 that is not available on NCBI. Small contigs were removed from the assembly prior to data simulation. During analysis, the human dataset used real ref read sequences rather than simulated reads.

TABLE 1

| Dataset | Reference | | Genome | | Long | | Ref | |
|---|---|---|---|---|---|---|---|---|
| name | genome | Accession | size | | reads | | reads | |
| Small | S. aureus | NC_007795.1 | 2.82 | Mbp | 56.4 | Mbp | 84.6 | Mbp |
| Medium | Human T2T | CP068256.2 | 51.3 | Mbp | 153 | Mbp | 1.54 | Gbp |
| | chr22 | | | | | | | |
| Large | Human | — | 3.1 | Gbp | 89.4 | Gbp | 105 | Gbp |

HG002

All datasets were simulated with long read lengths drawn from a normal distribution with mean 6 kbp and a mutation rate of 6%. Mutations were located at positions drawn uniformly at random throughout each long sequence and consisted strictly of transition mutations (A [symbol] G, C [symbol] T),

### Implementations of Rendering

Four alternative implementations of rendering methods were evaluated: original, minihyb, EM, and EM w/cons. These four methods represent a reduction to practice of various combinations of the methods presented above. A description of each method follows.

### Original

In the “original” method, the unmutated sequence reads and mutated sequence reads are mapped to a reference sequence. An initial pass of rendering then proceeds using the equations defined in Example 4: Adjustment for local sequence composition and unequal mutation rates, above.

In a subsequent pass of rendering, a k-mer comparison is used to further remove any residual error in the data. Briefly, k-mers present in each rendered read (“rendered k-mers”) are compared to the set of k-mers present in the unmutated sequence reads (“unmutated k-mers”). When a rendered read k-mer is not found in the set of unmutated k-mers, and is adjacent to a rendered k-mer that can be found in the unmutated k-mers, an attempt is made to change the k-mer that was not found so that it can be found in the unmutated k-mer set. The nucleotide in the first k-mer that is not covered by the 2nd k-mer is modified by applying a transition mutation, and if the resulting k-mer can then be found in the set of unmutated k-mers the change is maintained, otherwise it is reverted. The process is repeated on all adjacent k-mers in each rendered read until no more changes can be found that increase the number of rendered k-mers that are found in the unmutated k-mer set. The k-mer comparison process is similar to previously described methods for short read error correction, for example, those described in Greenfield et al., Blue: correcting sequencing errors using consensus and context, Bioinformatics, Volume 30, Issue 19, October 2014, Pages 2723-2732, doi.org/10.1093/bioinformatics/btu368.

minihyb

In the “minihyb” method, the unmutated sequence reads and mutated sequence reads are mapped to a reference sequence. Unmutated sequence reads that have a mapping location that overlaps a mutated sequence read trigger an ungapped re-alignment of the unmutated sequence read directly to the mutated sequence read. This direct unmutated: mutated read alignment can produce a better alignment in some situations, particularly where the sequence in the sample DNA diverges from the reference genome sequence. The unmutated: mutated read alignments are then subjected to soft-clipping using the well-known X-drop method, specifically if the alignment score drops by more than X from its peak, then the alignment following the position of the peak score is removed.

The next step of minihyb uses an ad-hoc method to select a set of high scoring alignments for use in the rendering calculation. The strategy applied in this step is to rank the alignments of unmutated sequence reads by their alignment score and select the top N alignments in non-overlapping windows of the dream sequence. When the alignment score is formulated using the approach of a substitution matrix and gap penalties, the score can have a probabilistic interpretation as the likelihood that the unmutated sequence read and mutated sequence read were generated by the same original sequence. Selecting the top N alignments effectively imposes a threshold on the probability that is dynamic, in that the threshold can scale up or down based on the density of mutations in a local region of the dream sequence.

The selected set of alignments are then used for rendering with the equations defined in Example 4: Adjustment for local sequence composition and unequal mutation rates.

### EM

In the “EM” method the unmutated sequence reads and mutated sequence reads are first mapped to a reference sequence. As with the minihyb method, unmutated sequence reads that have a mapping location that overlaps a mutated sequence read trigger an ungapped re-alignment of the unmutated sequence read directly to the mutated sequence read. Additional alignments are generated by direct alignment of unmutated sequence reads to mutated sequence reads using a short read aligner capable of multi-mapping the unmutated sequence reads onto multiple mutated sequence reads. When rendering a particular mutated sequence read, the set of all unmutated sequence read alignments onto that mutated sequence read are gathered, and then EM Method 1 is applied to carry out Expectation Maximization rendering.

### EM w/ Cons

The “EM w/cons” method applies Expectation Maximization with the additional step of generating consensus sequences for use in the unmutated: mutated sequence read alignment. The consensus sequences are generated as described above in the section titled “Alignment to a Reference Sequence and/or Consensus Sequence.” Once a set of alignments have been collected, the consensus of mutated sequence reads is discarded and rendering proceeds as described in EM Method 1.

### Evaluating Accuracy on Simulated Data

Simulated datasets were analyzed by providing the mutated sequence reads and the unmutated sequence reads to an analysis statistical method. This approach bypasses the step of assembling mutated sequence reads that would ordinarily be carried out in practical applications of this technology, and has the advantage of eliminating error in the assembly mutated sequence reads from our analysis of the accuracy of the rendering process.

Accuracy on the simulated datasets was evaluated by comparing rendered long sequences (representing the most probable sequence of the nucleic acid template) to the true unmutated long sequences that were generated by the simulation (representing the true nucleic acid template sequence). The comparison operates site-by-site, examining whether each site in the rendered sequence matches its corresponding site in the true sequence.

Where sites in the rendered sequence are identical to the true sequence the sites are classified as either True Positive or True Negative. A True Positive (TP) is a site that experienced a mutation, and that mutation was correctly identified by the renderer and the site was changed to match the original true sequence. A True Negative (TN) is a site that did not experience a simulated mutation, and was left unchanged by the rendering process.

Where sites in the rendered sequence are different to the corresponding site in the true sequence, they are classified as either False Positive or False Negative. A False Positive (FP) is a site that did not undergo a simulated mutation, but was changed by the rendering process so that it no longer matches the site in the true sequence. A False Negative (FN) is a site that underwent a simulated mutation, but the mutation was not correctly identified and resolved by the rendering.

Using the above definitions of TP, TN, FP, and FN, it becomes possible to calculate various measures of rendering performance, such as precision and recall. Per-base accuracy scores can be further stratified by the QV, for example, FP or FN where the QV is predicted to be very high or very low may be of interest. The results of the accuracy evaluation are presented below.

### Results on Simulated Data

As described in Table 1, three different simulated datasets were generated. Each dataset was analyzed using four different rendering methods described above: Original, minihyb, EM, and EM w/cons, except that there are no results for EM w/cons on the medium dataset because the dataset has only 3× coverage of mutated sequence reads, which is not sufficient to generate consensus sequences for much of the dataset. Results of the evaluations on simulated data are given in Table 2 below.

TABLE 2

| | Original | | | minihyb | | |
|---|---|---|---|---|---|---|
| | Precision | Recall | Emp. QV | Precision | Recall | Emp. QV |
| small | 0.995343 | 0.998809 | 35 | 1.0 | 0.99502 | 46 |
| medium | 0.997044 | 0.999169 | 37 | 0.99919 | 0.999891 | 50 |
| large | 0.971872 | 0.98707 | 23 | 0.990632 | 0.984398 | 28 |
| | EM | | | EM w/cons | | |
| | Precision | Recall | Emp. QV | Precision | Recall | Emp. QV |
| small | 0.999994 | 0.999748 | 49 | 0.999989 | 0.999970 | 57 |
| medium | 0.999917 | 0.999942 | 51 | — | — | — |
| large | 0.996425 | 0.997122 | 34 | 0.994323 | 0.994251 | 32 |

Several trends emerge from the simulated data. On all datasets the methods that use Expectation Maximization produce significantly better results than the simpler rendering methods. Second, the use of consensus sequence leads to a gain in accuracy on the small dataset, but not on the large dataset. The discrepancy could be explained by a difference in how the datasets were processed. Whereas the small dataset used the same genome sequence for both simulation of mutated sequence reads and the mapping of those mutated sequence reads during consensus generation, the large dataset used an assembly of the HG002 sample for simulation, but the hg38 human genome assembly for mutated sequence read mapping and consensus generation. It could be that the differences between the hg38 assembly and the HG002 assembly lead to poor consensus generation, which in turn degrades the accuracy of results.

Finally, accuracy on the small and medium datasets is somewhat higher than on the large dataset. There are several factors that could contribute to this observation. First, the large dataset used real sequence data rather than simulated data for the ref reads. Second, as noted above, the large dataset was processed using hg38 as a reference, rather than the HG002 genome from which the reads were simulated. It seems likely that the rendering methods which depend largely on reference genome mapping (original, minihyb) would be especially prone to error when significant differences exist between the reference genome and the simulation genome, leading to the relatively poor performance of those methods.

### Example 10: Accuracy Evaluation in Real Dataset

A selection of the rendering methods described above were subsequently applied to a real human genome dataset. Generation of the real human genome dataset is as described above. Application of the rendering method is the same as for simulation data, with simulated unmutated sequence reads replaced by standard whole genome sequencing (WGS) unmutated sequencing reads, and simulated mutated sequence reads replaced by real mutated sequence reads.

The advantage of evaluations on real datasets is that where a reasonably accurate prediction of the sequence of the original nucleic acid template is possible, the real datasets will capture effects of the sample prep and sequencing biochemistry which are difficult to simulate.

In total 22,945,744 mutated sequence reads were rendered, comprising 102.2 Gbp of sequence data. The mutated sequence read N50 in the dataset is 6.2 kbp. In the unrendered mutated sequence read data, approximately 5.4% of sites show a substitution difference relative to the hg38 reference genome sequence. Some of those differences are genuine differences between the HG002 sample and the hg38 reference sequence, while most are introduced mutations. In the rendered data, only 0.3% of sites show a substitution difference relative to the hg38 sample. Thus, the rendering process has greatly reduced the amount of difference relative to the reference.

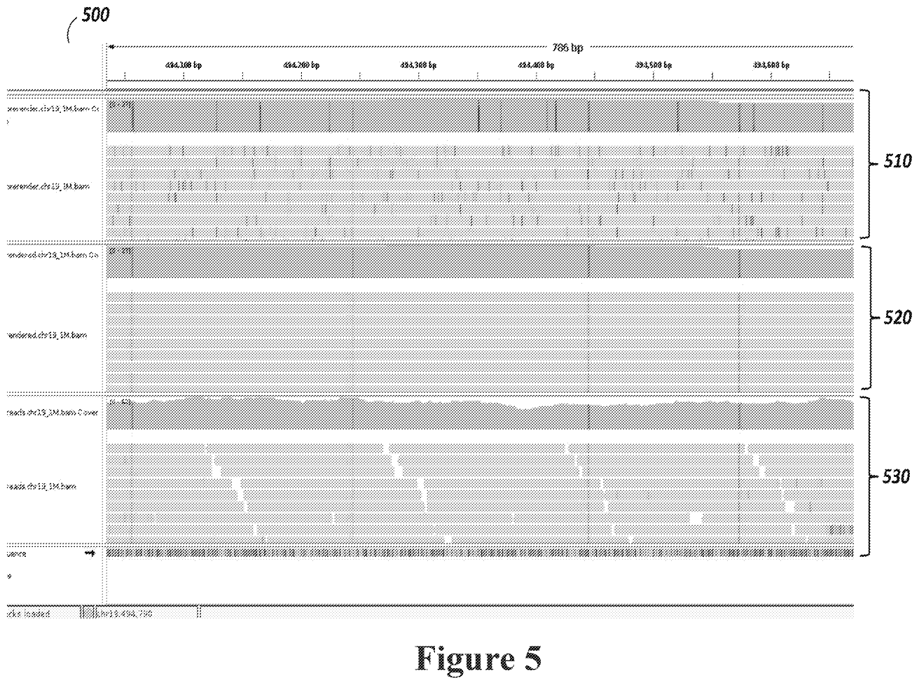

FIG. 5 shows a display 500 of the human genome data before and after the rendering process. In FIG. 5, three panels are shown in the display 500. In panels 510, 520, and 530, each sequence read is shown as a gray horizontal strip, where sites that are different compared to the hg38 reference sequence have been marked with a vertical bar. Panel 510 depicts mutated sequence reads of the HG002 sample, prior to rendering. A large number of differences relative to the hg38 reference sequence can be seen in the mutated sequence reads in panel 510. Panel 520 shows the same set of mutated sequence reads compared to the reference sequence after rendering with the minihyb method. As shown in panel 520, after rendering, the majority of the remaining differences between the rendered sequence and the reference sequence correspond to true variants in this sample, which true variants are shown in panel 530, depicting standard WGS unmutated sequence reads from a PCR-free library of the HG002 sample. Thus, it can be seen that prior to rendering, in panel 510, the mutated sequence reads contain a large number of differences (mutations) relative to the reference sequence. Rendering removes the majority of those differences, and the remaining differences are in agreement with the unmutated sequence reads (ref reads) as true single nucleotide variants (SNVs).

### Other Considerations

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (such as, feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 kbp to about 20 kbp should be interpreted to include not only the explicitly recited limits of from about 2 kbp to about 20 kbp, but also to include individual values, such as about 3.5 kbp, about 8 kbp, about 18.2 kbp, etc., and sub-ranges, such as from about 5 kbp to about 10 kbp, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods described herein may be embodied in a variety of other forms. Furthermore, various omissions, substitutions and changes in the methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A computer-implemented method of determining a sequence of a nucleic acid template by removing mutations found in a mutated sequence read, the method comprising:
a) aligning unmutated sequence reads of a nucleic acid template to mutated sequence reads, wherein the mutated sequence reads comprise one or more mutations, and
b) determining:
i) a most probable sequence of the nucleic acid template, and
ii) for each base of the most probable sequence, a per-base accuracy score correlated with the probability that the base matches with the nucleic acid template;
thereby determining the sequence of the nucleic acid template.

2. The method of claim 1, wherein the mutated sequence reads are of a mutated nucleic acid template.

3. (canceled)

4. The method of claim 1, wherein the one or more mutations comprise mutations which were randomly introduced during sample preparation by mutagenesis.

5-6. (canceled)

7. The method of claim 1, wherein i) comprises determining a most probable base for each site or a subset of sites associated with the alignment of the unmutated sequence reads to the mutated sequence reads.

8. (canceled)

9. The method of claim 1, wherein step b) comprises, on a site-by-site basis, summarizing the probability of each base at each site associated with the unmutated sequence reads.

10. The method of claim 1, wherein step b) is based on a probability of a mutation at each site of a mutated sequence read, a probability of an error associated with a sample preparation or sequencing process, variation in mutation rate between mutated sequence reads, or unequal mutation rates of adenine, cytosine, guanosine, and thymine.

11. (canceled)

12. The method of claim 1, wherein step b) comprises determining a most probable base for a site of a mutated sequence read that is not aligned to an unmutated sequence read, based on a statistically inferred probability of a mutation at the site.

13. (canceled)

14. The method of claim 1, wherein the nucleic acid template is derived from a nucleic acid sample comprising two or more copies of a repeat region of the nucleic acid template.

15. The method of claim 14, wherein step b) comprises estimating a probability that an unmutated sequence read was derived from a same copy of a repeat region as a mutated sequence read to which the unmutated sequence read is aligned.

16. The method of claim 14, wherein step b) comprises iteratively updating an estimate of the probability that an unmutated sequence read was derived from a same copy as the mutated sequence read to which the unmutated sequence read is aligned, while updating an inference of a most probable base.

17. (canceled)

18. The method of claim 16, wherein step b) is based on information that describes variations in read length.

19. The method of any of claims 14-17, wherein a contribution of each unmutated sequence read to a probability calculation of a most probable base is weighted by a weighting scheme related to sequence identity of the unmutated sequence read and a current iterative estimate of the sequence of the nucleic acid template.

20. (canceled)

21. The method of claim 14, wherein step b) comprises selecting a subset of unmutated sequence reads among unmutated sequence reads derived from different copies of the repeat region by scoring alignments of unmutated sequence reads to the mutated sequence reads and selecting top-scoring alignments.

22-23. (canceled)

24. The method of claim 14, wherein the two or more copies of a repeat region comprise two or more haplotypes, and wherein the method comprises determining a sequence of a haplotype of the nucleic acid sample.

25-26. (canceled)

27. The method of claim 1, wherein the method further comprises, prior to a), assembling the unmutated sequence reads, and wherein a) comprises aligning the mutated sequence reads to the assembly of unmutated sequence reads.

28. The method of claim 1, wherein the method further comprises, prior to a), assembling the mutated sequence reads, and wherein a) comprises aligning the unmutated sequence reads to the assembly of mutated sequence reads.

29. (canceled)

30. The method of claim 1, wherein the method further comprises, prior to a), assembling the mutated sequence reads, generating a consensus of the assembled mutated sequence reads, and aligning the unmutated sequence reads to the consensus, and further wherein step a) comprises replacing the consensus with the mutated sequence reads in said alignment of the unmutated sequence reads to the consensus.

31-32. (canceled)

33. The method of claim 4, wherein the mutations comprise substitutions, and wherein the substitutions comprise transition mutations.

34-35. (canceled)

36. The method of claim 1, wherein the method comprises:
aligning the unmutated sequence reads and the mutated sequence reads to a reference sequence;
determining an initial rendered sequence by determining a most probable base for each site associated with the alignment of the unmutated sequence reads to the mutated sequence reads, incorporating information that describes variation in mutation rate between mutated sequence reads or unequal mutation rates of adenine, cytosine, guanosine, and thymine; and
determining a further rendered sequence by comparing k-mers from the initial rendered sequence to k-mers from unmutated sequence reads.

37. The method of claim 1, wherein the method comprises:
aligning the unmutated sequence reads and the mutated sequence reads to a reference sequence;
realigning unmutated sequence reads that have a mapping location that overlaps a mutated sequence read directly to the mutated sequence read;
scoring alignments of unmutated sequence reads to the mutated sequence reads and selecting top-scoring alignments; and
determining a rendered sequence by determining a most probable base for each site associated with the alignment of the unmutated sequence reads to the mutated sequence reads.

38. The method of claim 1, wherein the method comprises:
determining a rendered sequence by:
determining a most probable base for each site associated with the alignment of the unmutated sequence reads to the mutated sequence reads, and applying an Expectation Maximation (EM) optimization process, wherein
the EM optimization process iteratively updates an estimate of the probability that an unmutated sequence read was derived from a same copy as the mutated sequence read to which the unmutated sequence read is aligned, while updating an inference of the most probable base.

39. The method of claim 1, wherein the method comprises:
generating a consensus of the assembled mutated sequence reads;
aligning the unmutated sequence reads to the consensus;
replacing the consensus with the mutated sequence reads in said alignment of the unmutated sequence reads to the consensus; and
determining a rendered sequence by:
determining a most probable base for each site associated with the alignment of the unmutated sequence reads to the mutated sequence reads, and
applying an Expectation Maximation (EM) optimization process, wherein the EM optimization process iteratively updates an estimate of the probability that an unmutated sequence read was derived from a same copy as the mutated sequence read to which the unmutated sequence read is aligned, while updating an inference of the most probable base.

40. A system comprising a processor configured to perform a method comprising:
a) aligning unmutated sequence reads of a nucleic acid template to mutated sequence reads of a mutated nucleic acid template, wherein the mutated sequence reads comprise one or more mutations, and
b) determining:
i) a most probable sequence of the nucleic acid template, and
ii) for each base of the most probable sequence, a per-base accuracy score correlated with the probability that the base matches with the nucleic acid template;
thereby determining the sequence of the nucleic acid template.

41. A computer readable medium comprising instructions that when executed by a processor perform a method comprising:
a) aligning unmutated sequence reads of a nucleic acid template to mutated sequence reads of a mutated nucleic acid template, wherein the mutated sequence reads comprise one or more mutations, and
b) determining:
i) a most probable sequence of the nucleic acid template, and
ii) for each base of the most probable sequence, a per-base accuracy score correlated with the probability that the base matches with the nucleic acid template;
thereby determining the sequence of the nucleic acid template.
